(19) United States
(12) Patent Application Publication
(10) Pub. No.: US 2022/0137045 A1
(43) Pub. Date: May 5, 2022

(54) METHOD FOR ESTIMATING THE EFFECTIVENESS OF TREATMENT WITH AN ANTI-CD20 AGENT IN A PATIENT WITH RHEUMATOID ARTHRITIS AND HAVING HAD AN INADEQUATE RESPONSE TO AT LEAST ONE BIOTHERAPY

(71) Applicant: SINNOVIAL, GRENOBLE (FR)

(72) Inventors: Philippe GAUDIN, MEYLAN (FR); Athan BAILLET, JARRIE (FR); Anaïs COURTIER, VOIRON (FR); Minh-Vu-Chuong NGUYEN, LE VERSOUD (FR); Jacques-Eric GOTTENBERG, STRASBOURG (FR)

(73) Assignee: SINNOVIAL, GRENOBLE (FR)

(21) Appl. No.: 17/607,474

(22) PCT Filed: Apr. 29, 2020
(86) PCT No.: PCT/FR2020/050723

§ 371 Date: Oct. 29, 2021

(30) Foreign Application Priority Data
May 3, 2019 (FR) 1904689

(51) Int. Cl. G01N 33/564

(52) U.S. Cl. CPC G01N 33/564; G01N 2333/70596; G01N 2800/60; G01N 2800/52; G01N 2800/102

(57) ABSTRACT

The present invention relates to a method for estimating the effectiveness of treatment with an anti-CD20 agent in a patient with rheumatoid arthritis and who has had an inadequate response to at least one prior biotherapy, consisting in analysing a biological sample of said patient for the expression of a set of biomarkers, the results of which make it possible to determine whether said agent is a treatment that will engender a beneficial response for said patient. The present invention also relates to a system for estimating the effectiveness of said treatment in said patient comprising means for measuring or receiving data, the expression level of said biomarkers and means for processing these data configured to estimate said effectiveness of said treatment in said patient.

### TECHNICAL FIELD

The present invention relates to the treatment of rheumatoid arthritis. It more particularly relates to a method for estimating the effectiveness of treatment with an anti-CD20 agent, in particular with Rituximab (RTX), in a patient with rheumatoid arthritis (RA) and having had an inadequate response to one or more prior biotherapy treatment(s), consisting in analysing a biological sample of said patient vis-à-vis the expression of a set of biomarkers, the correlation of the results obtained for this set of biomarkers making it possible, notably by comparison with reference values, to determine if the anti-CD20 agent is a promising treatment making it possible to lead to a beneficial response for said patient.

The present invention also relates to a system for estimating the effectiveness of said treatment in said patient comprising means for measuring or receiving data of the expression level of said biomarkers and means for processing these data configured to estimate said effectiveness of said treatment in said patient.

### TECHNICAL BACKGROUND

Rheumatoid arthritis (RA) is a chronic inflammatory disease characterised by synovitis, joint damage, functional handicap and a significant increase in mortality.

Early intervention using sDMARDs (synthetic Disease-Modifying Anti-Rheumatic Drugs) is now recognised as being essential for preventing structural joint damage and progressive loss of function. For patients that do not respond to treatment with sDMARDs or who develop an inadequate response to these drugs over time, bDMARDs (biological DMARDs) are one effective additional treatment option. In clinical practice, the first choice of biological therapy is normally a TNFα (Tumour Necrosis Factor alpha) inhibitor. Response to treatment can vary in a very significant manner from one patient to another. Around 30% to 40% of patients who start treatment with a TNFα inhibitor develop thereafter an insufficient or inadequate response to these drugs. The options for the continuation of treatment in TNFα insufficient patients comprise the use of a second biological agent. Given the number of bDMARD treatment options available for clinicians and their effectiveness in the treatment of RA, the passage between different bDMARDs is common practice. That is why, at the present time, the practitioner recommends, for most diseases, a first-line treatment then, in the event of insufficient or inadequate response, a second-line treatment, and so on. However, within this overall strategy, there exists a debate on the effectiveness relating to the use of another TNFα inhibitor (cycle) or a biological agent with a different mode of action (switching). In addition, the probability for the patient having already received an anti-TNFα of responding to another biological treatment decreases progressively as a function of the increasing number of failures of prior treatments. Thus, data from the literature indicate that any early intervention makes it possible to better contain the progression of the disease.

Unfortunately, today, the practitioner lacks objective elements capable of helping him in his therapeutic choice in order to target and identify a priori the suitable treatment for his patient. A tool capable of providing the clinician with a score of probability of response or non-response to a treatment would certainly be welcome.

Indeed, the time lost bringing to light a potential therapeutic failure is to the detriment of the effectiveness of the therapeutic action and the well-being of the patient, which in certain cases may have ended up in new symptoms or prejudicial and irreversible consequences in terms of general condition. In addition, they may be costly treatments which may be cumbersome to put in into practice, which is entirely unsatisfactory when a therapeutic failure is noted.

Considerable progress has been made over recent years concerning the diagnosis, care, treatment and follow-up of patients with chronic inflammatory diseases.

In terms of treatments of chronic inflammatory diseases, and notably chronic inflammatory rheumatisms, biotherapies notably exist which consist in biological molecules, such as proteins, antibodies, having a therapeutic action. Some of them are already used and others are under development.

Among chronic inflammatory diseases, rheumatoid arthritis is an auto-immune disorder of the synovia which is characterised by the proliferation of synoviocytes and the infiltration of inflammatory cells into the joint. Various cytokines play an important role in the regulation of inflammatory diseases.

bDMARDs targeting the tumour necrosis factor (TNF) or the co-stimulation of T-lymphocyte cells for example have enabled considerable progress for the treatment of RA. At present, 10 bDMARDs including the T-lymphocyte cell co-stimulation modulator Abatacept (ABA), anti-IL-6 Tocilizumab (TCZ) and Sarilumab (SAR), anti-CD20 Rituximab (RTX), anti-Interleukine-1 (IL-1) Anakinra (ANK) and anti-TNFα Adalimumab (ADA), Etanercept (ETN), Infliximab (IFX), Golimumab (GOL) and Certolizumab Pegol (CTP), are approved for the treatment of rheumatoid arthritis. However, the responses to each biological agent vary for each individual. Consequently, making an optimal choice of one or more bDMARD(s) within a therapeutic window of opportunity is essential to obtain effectiveness of treatment, which proves to be very expensive. Indeed, the chances of success of a biological treatment dwindle as a function of the increasing number of therapeutic failures with biotherapy.

The practitioner lacks however elements at his disposal to help him in his therapeutic choice. A tool capable of providing the clinician with a score of probability of response or non-response to a treatment would certainly be welcome.

In particular, there lacks at present very early biomarkers which can, among other things, provide guidance with regard to the possible response or non-response to a biological or conventional background treatment.

These biomarkers call on molecular biology and biochemistry. The hypothetico-deductive approach has reduced personalised medicine to several biomarkers, the interest of which has been fixed a priori and which has not made it possible to exhaust questions of early diagnosis or the theranostic approach. Thus, the search for the biomarker making it possible to predict the response to a biological treatment in chronic inflammatory diseases, and thus in RA, is an illusion. The multiplicity of genetic or biochemical biomarkers associated with the good clinical response or non-response to a biological treatment makes the task difficult.

Genomics, transcriptomics, epigenetics and proteomics are complementary and non-redundant pillars in this perspective.

A predictive approach of personalised or stratified medicine type is very novel in the field of chronic inflammatory rheumatisms and could make it possible to prescribe the right treatment to the right patient at the right moment, to limit the progression of the handicap by guiding the patient as quickly as possible to the treatment to which he has the greatest chance of responding, and to avoid prescribing treatments which, conversely, are associated with a low probability of response.

Rituximab is a chimeric monoclonal antibody directed against the molecule CD20 which binds specifically to this transmembrane antigen present on the surface of B cells from the pre-B stage to the mature B lymphocyte stage. The action of Rituximab induces B lymphocyte depletion according to different mechanisms, notably by cellular cytotoxicity or by apoptosis.

Some studies have focused on highlighting biomarkers making it possible to predict the response to RTX treatment in patients with RA. These studies essentially concern the characterisation of DNA and RNA biomarkers (Raterman et al., 2012), nevertheless these genes to not correspond to the relevant biomarkers according to the present invention; or even the characterisation of the population of specific circulating cells (Tony et al., 2015). However, DNA and RNA are subject to potential modifications (epigenetic, regulation of gene expression, splicing) linked to the environment before being translated into proteins which are the final effectors. The proteomic approach thus makes it possible to minimise possible variations between the expression level of biomarkers and the clinical results observed.

Studies have shown a link between the presence of anti-citrullinated peptide antibodies and the rheumatoid factor at the basal level with a better response to an RTX treatment (Isaacs et al., 2013). The serum concentrations of S100A8A9 and IL-33 are predictors of the response to Rituximab in biotherapy naive RA patients (Choi et al., 2015a; Sellam et al., 2016). However, these studies have not used a strategy of combination of biomarkers to improve the specificity or the sensitivity and focus on biotherapy naive RA patients. In addition, these studies focus on biotherapy naive RA patients or belonging to a non-differentiated population of naive patients and patients in rotation, that is to say who have had an insufficient or inadequate response to at least one biotherapy. The distinction between so-called naive populations and those having already received one or more biotherapies is very important because numerous studies have demonstrated a change of the proteome after treatment with biotherapies (Takeuchi et al., 2007). Thus, biomarkers predictive of the response in biotherapy naive patients are capable of being modified and to be no longer relevant in patients having already received one or more biotherapies. Also, the distinction of the therapeutic situation of the patient (naive or in rotation) is an essential criterion in the choice of predictive biomarkers. Whereas most studies on RTX concerning patients in rotation situation, that is to say who have had an insufficient or inadequate response to at least one biotherapy, concentrate on the effectiveness of RTX and its therapeutic relevance compared to other bDMARDs (Soliman et al., 2012), the present invention focuses on the prediction of response of a population of patients with rheumatoid arthritis and having had an insufficient or inadequate response to at least one biotherapy.

There thus exists a need to identify novel methods and/or biomarkers making it possible to guide the practitioner in a personalised manner towards the treatment that is the most promising in terms of effectiveness for a given patient with a chronic inflammatory disease, in particular for patients suffering from rheumatoid arthritis, and notably for those who are in a situation of inadequate response to one or more biotherapy treatment(s).

The present invention responds to this technical problem vis-à-vis the response to a treatment with an anti-CD20 agent for a patient with rheumatoid arthritis not having had a sufficient therapeutic response to one or more prior biotherapy treatment(s); the inventors having identified a set of biological biomarkers of which the expression level detected in a biological sample from such a patient makes it possible to estimate the probability of an effective response to this treatment in this patient.

### SUMMARY OF THE INVENTION

The present invention relates to a method for estimating the effectiveness of treatment with an anti-CD20 agent in a patient with rheumatoid arthritis and not having had an adequate therapeutic response to one or more prior biotherapy treatment(s), said method comprising:
a) the in vitro measurement of the expression level of at least two biomarkers chosen from the group consisting of Fetuin-A (FetA), Thyroxine Binding Globulin (TBG), Serum Amyloid A protein (SAA), C4b Binding Protein (C4BP), Protein complex S100A8/A9 (S100A8A9), Lipoprotein(a) (Lp(a)), Haptoglobin (HAPT), Complement protein C1q (C1q), and C-reactive protein (CRP) in a biological sample from said patient,
b) the estimation of said effectiveness of treatment with said anti-CD20 agent in said patient as a function of each expression level measured for a biomarker chosen from said group.

In particular, the method for estimating the effectiveness of treatment with an anti-CD20 agent according to the invention comprises:
a) the in vitro measurement of the expression level of at least two biomarkers chosen from the group consisting of: Fetuin-A (FetA), Thyroxine Binding Globulin (TBG), Serum Amyloid A protein (SAA), C4b Binding Protein (C4BP), Protein complex S100A8/A9 (S100A8A9), Lipoprotein(a) (Lp(a)), Haptoglobin (HAPT), Complement protein C1q (C1q), and C-reactive protein (CRP) in a biological sample from said patient,
b1) the comparison of the expression level measured at step a) compared to that measured in a plurality of samples of patients with rheumatoid arthritis and having received a treatment with said anti-CD20 agent for which the effectiveness of treatment is known; said comparison being carried out using a statistical learning model using as input data the expression levels of at least two of the biomarkers measured at step a),
b2) the estimation of said effectiveness of treatment with said anti-CD20 agent in said patient as a function of the results determined by the model defined at step b1).

The set of biomarkers identified by the inventors is thus particularly suited to estimating the effectiveness of treatment with an anti-CD20 agent in a patient with rheumatoid arthritis.

The present invention furthermore relates to a system for estimating the effectiveness of treatment with an anti-CD20 agent in a patient with rheumatoid arthritis and who has had an inadequate response to at least one prior biotherapy treatment, said system comprising:
means for measuring or receiving measurement data of the expression level of at least two biomarkers chosen from a group consisting of: Fetuin-A (FetA), Thyroxine Binding Globulin (TBG), Serum Amyloid A protein (SAA), C4b Binding Protein (C4BP), Protein complex S100A8/A9 (S100A8A9), Lipoprotein(a) (Lp(a)), Haptoglobin (HAPT), Complement protein C1 q (C1q), C-reactive protein (CRP) in a biological sample from said patient,
means for processing measurement data configured to estimate said effectiveness of treatment in said patient as a function of each expression level measured for a biomarker chosen from this group.

Preferably, the estimation method and the estimation system according to the invention make it possible to estimate the response to an anti-CD20 agent, in particular an agent that binds, directly or indirectly, to the molecule CD20 to lead to the destruction of B lymphocytes. Advantageously, the estimation method and the estimation system according to the invention make it possible to estimate the response to an antibody leading to the destruction of B lymphocyte carriers of CD20, and in particular to estimate the response to Rituximab.

### BRIEF DESCRIPTION OF THE FIGURES

FIG. 1 represents the ROC (Receiver Operating Characteristic) curve obtained during the evaluation of the performances of the method according to the invention with the study of the 3 biomarkers Fet-A, TBG and Lp(a) for the prediction of the response to RTX. It represents an example of the sensitivity of the test (Y-axis) as a function of the complementary of the specificity of the test: 1−specificity (X-axis).

### DETAILED DESCRIPTION OF THE INVENTION

The problem encountered in the field of the invention for the development of a robust predictive test firstly consists in identifying the biomarkers which, taken together, make it possible to obtain a relevant prediction with both high specificity and high sensitivity.

That is why, according to a first aspect, the present invention relates to a method for estimating the effectiveness of treatment with an anti-CD20 agent in a patient with rheumatoid arthritis and who has had an inadequate response to at least one prior biotherapy treatment, said method comprising, or even consisting in:

a) the in vitro measurement of the expression level of at least two biomarkers chosen from the group consisting of Fetuin-A (FetA), Thyroxine Binding Globulin (TBG), Serum Amyloid A protein (SAA), C4b Binding Protein (C4BP), Protein complex S100A8/A9 (S100A8A9), Lipoprotein(a) (Lp(a)), Haptoglobin (HAPT), Complement protein C1q (C1q), and C-reactive protein (CRP) in a biological sample from said patient,
b) the estimation of said effectiveness of treatment with said anti-CD20 agent in said patient as a function of each expression level measured for a biomarker chosen from said group.

The inventors have in fact identified sets or combinations of relevant biomarkers for estimating the effectiveness of treatment with an anti-CD20 agent in a patient with rheumatoid arthritis and who has had an inadequate response to at least one prior biotherapy treatment, namely at least two biomarkers chosen from the group consisting of Fetuin-A (FetA), Thyroxine Binding Globulin (TBG), Serum Amyloid A protein (SAA), C4b Binding Protein (C4BP), Protein complex S100A8/A9 (S100A8A9), Lipoprotein(a) (Lp(a)), Haptoglobin (HAPT), Complement protein C1q (C1q), and C-reactive protein (CRP).

In a particular, the method for estimating the effectiveness of treatment with an anti-CD20 agent according to the invention comprises:

a) the in vitro measurement of the expression level of at least two biomarkers chosen from the group consisting of: Fetuin-A (FetA), Thyroxine Binding Globulin (TBG), Serum Amyloid A protein (SAA), C4b Binding Protein (C4BP), Protein complex S100A8/A9 (S100A8A9), Lipoprotein(a) (Lp(a)), Haptoglobin (HAPT), Complement protein C1q (C1q), and C-reactive protein (CRP) in a biological sample from said patient,
b1) the comparison of the expression level measured at step a) compared to that measured in a plurality of samples of patients with rheumatoid arthritis and having received a treatment with said anti-CD20 agent for which the effectiveness of treatment is known; said comparison being carried out using a statistical learning model using as input data the expression levels of at least two of the biomarkers measured at step a),
b2) the estimation of said effectiveness of treatment with said anti-CD20 agent in said patient as a function of the results determined by the model defined at step b1).

The measurement of the expression level of particular combinations of these particular biomarkers and their analysis notably by means of a statistical learning model make it possible to obtain a relevant estimation of the prediction of response to an anti-CD20 agent for a patient with rheumatoid arthritis.

The present invention thus relates to a personalised method for predicting the response to an anti-CD20 agent, in particular to Rituximab, of a given patient with rheumatoid arthritis and who has had an inadequate response to at least one prior biotherapy treatment. It makes it possible to determine, in a patient with rheumatoid arthritis and who has had an inadequate response to at least one prior biotherapy treatment, a level of effectiveness based on a probability of good response and non-response to this anti-CD20 agent. The method according to the invention thus makes it possible to identify responsive patients and in particular patients non-responsive to the anti-CD20 agent in question.

As regards step a) of the method according to the invention, the relevant biomarkers identified by the inventors are defined hereafter.

Fetuin A, also known as Alpha 2-Heremans Schmid Glycoprotein (AHSG), is a so-called circulating “carrier” protein secreted by the liver abundantly present in the foetus (Dabrowska et al., 2015). Fetuin A may influence the resolution of inflammation by modulating phagocytosis of apoptotic cells by macrophages. Several studies show the expression level of FetA is different in RA patients compared to that of healthy subjects whereas certain studies show a reduced expression of FetA (Papichev et al., 2018) in RA, while others observe conversely an increase (Harman et al., 2017). To the knowledge of the inventors, the predictive aspect of FetA as biomarker in the treatment of RA by bDMARDs has not been reported in the literature.

Thyroxine Binding Globulin (TBG) (Refetoff et al., 1996) binds circulating thyroid hormones; it ensures the transport of T3 (triiodothyronine) and 70% of T4 (thyroxine). TBG concentration variations intervene directly on the amount of bioavailable thyroid hormones. To the knowledge of the inventors, no study describes the role of TBG in RA and its predictive potential in the response to bMARD treatments.

Serum Amyloid A proteins (SAA) (Xu et al., 2005) belong to a family of proteins associated with the acute phase of inflammation. Expressed at a basal level, they are synthesised during the acute phase, at levels of 100 to 1000 times their normal value, mainly produced by the liver under the action of pro-inflammatory cytokines. It has been suggested that SAA levels better reflect the activity of the disease in joint inflammatory diseases than the sedimentation rate and CRP traditionally used to estimate the disease activity score in RA. One study shows that the concentration of SAA remains high during treatment with DMARD unlike conventional RA indicators (CRP and sedimentation rate) and that it could thus be a more sensitive biomarker for determining the activity of the disease. A correlation between the basal level of SAA and RA activity has been described with a decrease in the SAA level one year after treatment with a bDMARD (Adalimumab, Infliximab, Etanercept or Anakinra (Connolly et al., 2012)). These studies highlight the relationship between the decrease in the SAA level in the course of treatment and the associated clinical response but does not focus on the predictive value of the basal level of SAA in itself. One study has however observed a reduced expression of SAA at the basal level in responders to Rituximab but which remains constant throughout treatment (Raterman et al., 2013). However, the predictive character of SAA for Rituximab treatment has not been broached either alone or in the form of a combination. In addition, this study concerns a mixed population of naive patients or patients in rotation.

The complement system (Ricklin and Lambris, 2013) is one of the defence mechanisms that intervenes in the destruction of infectious agents, in the elimination of immune complexes, but also in the control of inflammatory responses and the modulation of specific immune responses. It is an activation cascade and the fraction C3 plays a central role in the activation of the complement since all of the activation pathways of the complement end up in its cleavage by specific proteolytic systems in C3a and C3b. A link between the complement system and RA has been highlighted. The excessive or poorly orientated activation of the complement contributes to the pathogenesis of inflammatory diseases such as rheumatoid arthritis (Swaak et al., 1987). Complement binding protein C4b (C4BP) is a protein involved in the complement system where it acts as inhibitor. It inhibits the action of conventional pathways and lectins, more particularly C4. It also has the capacity of binding C3b. The use of recombinant protein C4BP in two RA mouse models reduces severity of the disease (Blom et al., 2009). To the knowledge of the inventors, no study describes the predictive potential of C4BP in the response to bMARD treatments nor in particular for Rituximab.

Complement protein C1q (C1q) is the unit that recognises activators of the conventional pathway of the complement system. It forms part of the fraction C1 of the complement (two sub-units C1q and a tetramer formed of two molecules of C1r and two molecules of Cis). The fractions C1, C4 and C2 interact to form the C3 convertase of the conventional activation pathway of the complement responsible for the activation of C3. Anti-citrullinated protein antibodies (ACPA), known to play a role in RA, activate in vitro the complement system via the conventional pathway (Trouw et al., 2017). The plasma level of the complex C1q-C4 is higher in patients with active RA compared with those in remission (Wouters et al., 2006). A former study on the other hand does not show any correlation between the serum level of C1q with the known activity parameters of RA (Persellin and Cheng, 1981). To the knowledge of the inventors, no study describes the predictive potential of C1q in the response to bMARD treatment nor in particular for Rituximab.

S100A8 (also called MRP8, calgranulin A) and S100A9 (MRP14, calgranulin B) are calcium binding proteins (EF hand motifs) belonging to the S100 protein family. The protein complexes S100A8/A9 (calprotectin) are inflammatory proteins mainly secreted by polynuclear neutrophils and described since several years as actors in the development of chronic inflammatory rheumatisms (Baillet, 2010; Baillet et al., 2010). This complex has been described as a parameter correlated with RA activity but also the estimation of its basal level may be predictive of the response to methotrexate in RA patients (Patro et al., 2016). S100A8/A9 proteins also appear as biomarkers for the response to Etanercept biotherapy (Drynda et al., 2004) but may have a predictive character of response to bDMARDs such as anti-TNFα (Choi et al., 2015b) or Rituximab (Nair et al., 2016). However these two studies coming from the same cohort concern a mixed population of naive patients and patients in rotation.

To the knowledge of the inventors, no study describes the predictive potential of Calprotectin in the response to bMARD treatments nor in particular for Rituximab for a population of RA patients in rotation.

Lipoprotein(a) (Lp(a)) (Orsó and Schmitz, 2017) is a lipoprotein constituted of LDL Low Density Lipoprotein and apo B100 of which the metabolism is independent of the other lipidic fractions. A high plasmatic concentration of Lp(a) is an independent risk factor for the development of atherosclerosis, with risk of cardiovascular and cerebrovascular accidents. A decrease in the expression of Lp(a) is observed in the course of treatment of RA patients with Tocilizumab or Adalimumab, not just in responders but also non-responders (Gabay et al., 2016). However, this drop in Lp(a) consecutive to an anti-TNFα treatment is not found in other studies (Seriolo et al., 2006). To the knowledge of the inventors, no study describes the predictive potential of Lp(a) in the response to bMARD treatments nor in particular for Rituximab.

Haptoglobin (Hp) (Andersen et al., 2017), synthesised by the liver, is a circulating protein which easily combines with extra-globular haemoglobin to carry out its elimination. It is constituted of two alpha chains and two beta chains. Its level decreases considerably in the case of destruction of red blood cells and represents a haemolysis marker. It is also an inflammatory reaction protein. Its expression is altered in RA patients compared to that of healthy subjects (Saroha et al., 2011). An increase in its basal serum level may be predictive of non-response to Methotrexate in RA patients (Tan et al., 2016). The combination with vitamin D binding protein and apolipoprotein C-III has a predictive potential of the response to an anti-TNFα (Etanercept) in bDMARD naive RA patients (Blaschke et al., 2015). To the knowledge of the inventors, no study describes the predictive potential of haptoglobin in the response to Rituximab.

C-reactive protein (CRP), is a marker of the inflammatory status, the clinical use of which is very widespread and notably in the monitoring of chronic inflammatory rheumatisms (Amos et al., 1977). Its expression increases sharply in acute phase. These cytokines, in their turn, signal stromal cells for the synthesis of secondary inflammatory mediators such as interleukin 6 (IL-6), interleukin 8 (IL-8) or instead monocyte chemoattractant proteins. CRP is traditionally used to estimate the RA activity score (Wells et al., 2009).

Preferably, the method for estimating the effectiveness of treatment according to the invention is based on the in vitro measurement of the expression level of at least three of the aforementioned nine biomarkers, of at least four, of at least five, of at least six, of at least seven, of at least eight, or nine biomarkers of the group consisting of Fetuin-A (FetA), Thyroxine Binding Globulin (TBG), Serum Amyloid A protein (SAA), C4b Binding Protein (C4BP), Protein complex S100A8/A9 (S100A8A9), Lipoprotein(a) (Lp(a)), Haptoglobin (HAPT), Complement protein C1q (C1q), and C-reactive protein (CRP).

The preferred embodiments of the method for estimating the effectiveness of treatment according to the invention comprises the in vitro measurement of the expression level of particular combinations of the following biomarkers among the list of aforementioned nine biomarkers:
at least Fet-A and TBG or at least TBG and SAA, in a more preferred manner at least Fet-A and Lp(a);
at least Fet-A, SAA and Lp(a) or at least Fet-A, TBG and C4BP, or further and in a more preferred manner at least Fet-A, TBG and Lp(a) or at least Fet-A, C4BP and Lp(a).

These sets make it possible to obtain the most relevant results in terms of estimation of the effectiveness of treatment.

With these combinations of the aforementioned two or three preferred biomarkers, it is also possible in particular to measure in vitro the expression level of at least one other biomarker chosen from the remaining six or seven in the list of nine biomarkers described in the present description, namely Fetuin-A (FetA), Thyroxine Binding Globulin (TBG), Serum Amyloid A protein (SAA), C4b Binding Protein (C4BP), Protein complex S100A8/A9 (S100A8A9), Lipoprotein(a) (Lp(a)), Haptoglobin (HAPT), Complement protein C1q (C1q), and C-reactive protein (CRP). The method according to the invention makes it possible to estimate the effectiveness of treatment with an anti-CD20 agent in a patient with rheumatoid arthritis who has had an inadequate response to at least one prior biotherapy treatment.

“Patient with rheumatoid arthritis who has had an inadequate response” is taken to mean a patient with rheumatoid arthritis who has had an insufficient response to at least one prior biotherapy treatment, but also a patient with rheumatoid arthritis who has had a satisfactory response to at least one prior biotherapy treatment but who has presented at least one adverse event of moderate or severe intensity during prior treatment(s) necessitating the stoppage of the treatment.

“Insufficient response to at least one prior biotherapy treatment” is taken to designate a patient not having presented a positive therapeutic response to one or more prior treatment(s) with a bDMARD (biological Disease-Modifying Anti-Rheumatic Drug). Within the scope of the treatment of chronic inflammatory diseases, current therapeutic strategies are conducted to reduce the activity of the rheumatism and the response to the treatment is assessed during the first year, generally after 6 months. Concerning rheumatoid arthritis, the response to treatment is determined by the evolution of the activity of the rheumatism according to the EULAR (European League Against Rheumatism) response. The EULAR response takes into account the activity of the rheumatism which is evaluated by the DAS28 (Disease Activity Score 28) as well at its variation. The DAS is a composite score calculated on the basis of the number of painful joints out of 28 joints, VAS (Visual Analogue Scale), and a biological inflammatory parameter: SR (sedimentation rate) or CRP (Prevoo et al., 1995). The EULAR response at a time T is defined as a function of the DAS28 score at time T and the difference between the DAS28 at time T and the initial DAS28, that is to say before treatment. Within the scope of the present invention, “insufficient response to a treatment” is taken to mean in particular a EULAR response with a DAS28 at time T greater than 3.2 or a variation in DAS28 between time T and the DAS28 before treatment less than or equal to 1.2.

Conversely, “sufficient response to a treatment” is taken to mean a EULAR response with a DAS28 at time T less than or equal to 3.2 associated with a variation in DAS28 between time T and before treatment greater than 1.2.

Biotherapy is taken to mean a therapy resorting to the use of a bDMARD. DMARDs are a category of drugs defined by their use in rheumatoid arthritis to slow down the progression of the disease. Several types of DMARD exist, classed in the following manner:
synthetic DMARDs (sDMARDs) which comprise conventional synthetics (csDMARDs) and targeted synthetics (tsDMARDs). csDMARDs are traditional drugs such as methotrexate, sulfasalazine, leflunomide, hydroxychloroquine, gold salts, etc. tsDMARDs are drugs which have been developed to target a particular molecular structure
biological DMARDs (bDMARDs) which comprise original biological DMARDs (boDMARDs) and biosimilar DMARDs (bsDMARDs). bsDMARDs are those which have the same primary, secondary and tertiary structure as the original biological treatment (boDMARD) and have an effectiveness and safety similar to those of the original protein.

“Sufficient response to a treatment” is taken to designate a patient having presented a sufficient therapeutic response to one or more prior treatment(s) by a bDMARD (biological Disease-Modifying Anti-Rheumatic Drug). Within the scope of the present invention, “sufficient response to a treatment” is in particular taken to mean a EULAR response with a DAS28 after 6 months less than or equal to 3.2 associated with a variation of DAS28 after 6 months and of DAS28 before treatment greater than 1.2.

“Adverse event”, or AE, is taken to mean any untoward medical occurrence in a patient, whether this occurrence is linked or not to the biotherapy treatment. If this adverse event is considered by the physician as having a scientifically reasonable causality link with the procedure, the method, the act or the treatment, it is qualified as adverse effect. The expression “scientifically reasonable causality link” signifies that there exists proof or an argument making it possible to suggest, in scientific terms, a cause and effect relationship between the untoward and adverse reaction and the procedure, the method, the act or the treatment.

The intensity of adverse events is evaluated by the physician using the following classification, well-known in the field:
grade 1 mild intensity: adverse event generally transitional and not interfering with everyday activities
grade 2 moderate intensity: adverse event sufficiently discomforting to interfere with normal everyday activities
grade 3 severe intensity: adverse event considerably modifying the normal course of activities of the subject, or invalidating, or constituting a threat to the life of the subject.

The grades of all known adverse events as a function of pathologies are listed by the National Cancer Institute and accessible on the web site of the National Institutes of Health (Common Terminology Criteria for Adverse Events (CTCAE); https://safetyprofiler-ctep.nci.nih.gov/CTC/CTC.aspx). The different adverse events linked to a biotherapy treatment are notably classed in the Summary of Product Characteristics (SmPC).

Preferably, the method according to the invention makes it possible to estimate the effectiveness of treatment with an anti-CD20 agent which is Rituximab in a patient with rheumatoid arthritis and who has had an inadequate response to at least one prior anti-TNFα biotherapy treatment.

In an even more preferred manner, the method according to the invention makes it possible to estimate the effectiveness of treatment with an anti-CD20 agent in a patient with rheumatoid arthritis who has had an inadequate response to at least one prior treatment chosen from among Etanercept, Adalimumab, Infliximab, Tocilizumab, Abatacept, Certolizumab Pegol, Golimumab, Sarilumab and Anakinra, and preferably to a single one of these treatments and in particular to Etanercept, Adalimumab, Infliximab, Certolizumab Pegol or Golimumab.

According to the invention, the method makes it possible to estimate the effectiveness of treatment with an anti-CD20 agent. Such an agent may be defined as being an agent that is capable of binding, directly or indirectly, to the surface molecule CD20, in particular leading to a destruction of B lymphocytes. Among these agents, the bDMARD Rituximab may notably be cited.

In a particularly advantageous manner, the method according to the invention makes it possible to estimate the effectiveness of treatment with an anti-CD20 agent which is Rituximab.

Any biological sample constituted of a biological fluid may be used within the scope of the invention for measuring in vitro the expression level of the aforementioned biomarkers and combinations of biomarkers, and among which may notably be cited synovial fluid, serum, plasma, saliva, urine, etc., preferably serum.

According to a preferred embodiment, the expression level of the aforementioned biomarkers and combinations of biomarkers is measured in vitro on a sample of serum from the patient for whom it is sought to estimate the effectiveness of treatment with an anti-CD20 agent which is Rituximab.

Advantageously, the method for estimating the effectiveness of treatment according to the invention comprises at step a) the in vitro measurement of the expression level of the biomarkers or combinations of protein biomarkers.

Particularly preferred embodiments of the method according to the invention are the following, each being to apply to the combinations of biomarkers defined previously, namely at least two biomarkers chosen from the group consisting of Fetuin-A (FetA), Thyroxine Binding Globulin (TBG), Serum Amyloid A protein (SAA), C4b Binding Protein (C4BP), Protein complex S100A8/A9 (S100A8A9), Lipoprotein(a) (Lp(a)), Haptoglobin (HAPT), Complement protein C1 q (C1q), and C-reactive protein (CRP):
The estimation of the effectiveness of treatment with Rituximab, in a patient with rheumatoid arthritis;
The estimation of the effectiveness of treatment with Rituximab, in a patient with rheumatoid arthritis comprising the measurement of the protein expression level of at least two markers chosen from the nine mentioned in the present description;
The estimation of the effectiveness of treatment with Rituximab, in a patient with rheumatoid arthritis and who has had an inadequate response to at least one prior biotherapy treatment chosen from Etanercept, Adalimumab, Infliximab, Tocilizumab, Abatacept, Certolizumab Pegol, Golimumab, Sarilumab and Anakinra, preferably to a single one of these treatments;
The estimation of the effectiveness of treatment with Rituximab, in a patient with rheumatoid arthritis and who has had an inadequate response to at least one prior biotherapy treatment chosen from Etanercept, Adalimumab, Infliximab, Tocilizumab, Abatacept, Certolizumab Pegol, Golimumab, Sarilumab and Anakinra, comprising the measurement of the protein expression level of at least two markers chosen from the nine mentioned in the present description, preferably to a single one of these treatments;
The estimation of the effectiveness of treatment with Rituximab, in a patient with rheumatoid arthritis and who has had an insufficient response to at least one prior biotherapy treatment chosen from Etanercept, Adalimumab, Infliximab, Tocilizumab, Abatacept, Certolizumab Pegol, Golimumab, Sarilumab and Anakinra, preferably to a single one of these treatments;
The estimation of the effectiveness of treatment with Rituximab, in a patient with rheumatoid arthritis and who has had an insufficient response to at least one prior biotherapy treatment chosen from among Etanercept, Adalimumab, Infliximab, Tocilizumab, Abatacept, Certolizumab Pegol, Golimumab, Sarilumab and Anakinra, comprising the measurement of the protein expression level of at least two markers chosen from the nine mentioned in the present description, preferably to a single one of these treatments.

At step b1) of the estimation method according to the invention, the expression level of the biomarkers or combinations of biomarkers measured at step a) described above is compared with that measured in a plurality of samples of patients with rheumatoid arthritis and having received a treatment with an anti-CD20 agent for which the effectiveness of treatment is known.

This comparison is carried out by means of a statistical learning model using as input data the expression levels of at least two of the biomarkers measured at step a). To do so, any statistical learning model may be used, and notably the models obtained by logistic regression methods, discriminant analysis, neural networks, decision tree learning, support vector machines (SVM), or aggregation of models.

Preferably, in the method for estimating the effectiveness of treatment with an anti-CD20 agent according to the invention, the expression levels of each biomarker measured at step a) are used to obtain a score linked to the estimation of the effectiveness of treatment in said patient, said score being compared with at least one predetermined threshold so as to classify the prognosis among a plurality of classes. In this embodiment, it is notably possible to use a plurality of classes which comprises at least two classes of which one class of non-response to the treatment with said anti-CD20 agent, and preferably which comprises, or even consists in, two classes of which one class of non-response to the treatment with said anti-CD20 agent. The two classes may for example be derived from so-called “non-responder” patients, who present an insufficient response to the treatment and so-called “responder” patients who present a sufficient response to the treatment. Still in this embodiment, the estimation of the effectiveness of treatment in the patient with rheumatoid arthritis and who has had an inadequate response to at least one prior biotherapy treatment comprises the comparison of said score with a predetermined threshold below which poor effectiveness is predicted and above which good effectiveness is predicted.

Preferably, the method for estimating the effectiveness of treatment with an anti-CD20 agent according to the invention uses at step b1) a learning model based on a prior analysis of samples of a cohort comprising patients treated with said anti-CD20 agent presenting good responses to the treatment and patients treated with said anti-CD20 agent presenting poor responses to the treatment. In this embodiment, the learning model is preferably based on a prior analysis which comprises the application of a method for learning and selecting variables. Advantageously, logistic regression will be used as method for learning and selecting variables.

Still in this embodiment based on a prior analysis which comprises the application of a method for learning and selecting variables, the expression levels of the biomarkers or combinations of biomarkers measured at step a) are weighted as a function of the prior analysis of the cohort comprising patients treated with said anti-CD20 agent presenting good responses to the treatment and patients treated with said anti-CD20 agent presenting poor responses to the treatment to derive the score linked to the estimation of the effectiveness of treatment.

Still in this embodiment based on a prior analysis which comprises the application of a method for learning and selecting variables, the method for estimating the effectiveness of treatment according to the invention may use a method of learning by decision tree. According to this embodiment, the levels of expression of the biomarkers or combinations of biomarkers measured at step a) are compared with a reference value at each node of the tree.

The reference values may be obtained by the analysis of the expression level of the biomarkers or combinations of biomarkers in biological samples of a set of patients with rheumatoid arthritis before treatment so as to have available a set of data on the expression levels of the biomarkers associated with each biological sample of each patient.

These reference values can change over time as a function of the results obtained with other patients that complete the number of results serving to define the threshold value.

According to a second aspect, the invention also relates to a system for estimating the effectiveness of treatment with an anti-CD20 agent in a patient with rheumatoid arthritis and who has had an inadequate response to at least one prior biotherapy treatment, said system comprising:
means for measuring or receiving measurement data of the expression level of at least two biomarkers chosen from a group consisting of: Fetuin-A (FetA), Thyroxine Binding Globulin (TBG), Serum Amyloid A protein (SAA), C4b Binding Protein (C4BP), Protein complex S100A8/A9 (S100A8A9), Lipoprotein(a) (Lp(a)), Haptoglobin (HAPT), Complement protein C1q (C1q), and C-reactive protein (CRP) in a biological sample from said patient,
means for processing measurement data configured to estimate said effectiveness of treatment in said patient as a function of each expression level measured for a biomarker chosen from this group.

Among the means for measuring the expression level of the selected biomarkers or combinations of biomarkers, it is notably possible to cite specific reagents of each of the biomarkers such as enzymes, substrates or antibodies which may be used in methods, such as among others nephelometry, chemoluminescence, immunoturbidimetry, flow cytometry, ELISA, etc, but also physical means such as mass spectrometry analysis methods for example.

The system according to the invention may moreover contain means for receiving measurement data, thus making it possible to estimate, for said patient, the effectiveness of response to the treatment with the anti-CD20 agent, from data supplied for example by a practitioner having had the expression level of biological biomarkers measured such as described previously.

The reception means may notably comprise transmission/reception means for exchanging with a remote server through a communication network such as an intranet network or the secure internet network. The device may also comprise input means such as a keyboard.

The data processing means may notably call on database management, code instructions, the development of software comprising an algorithmic brick, an interface to enable the user to consult the results, etc. These different elements may be recorded on a storage support such as a hard disc, a CD ROM, a USB key, or any other storage support known to those skilled in the art.

They may be implemented by a device which may be fixed or mobile. The device is for example a personal computer, a mobile telephone, an electronic tablet, or any other type of terminal known to those skilled in the art.

In an alternative, the system may also comprise transmission means for transmitting, still via intranet or internet for example, the results of the estimation of the effectiveness of the treatment in the patient concerned.

According to another advantageous alternative, the system according to the invention comprises means for receiving the effectiveness data obtained, and does so in order to complete and enrich the reference values in view of the result of treatment obtained with regard to the expression level of the selected biomarkers.

### Examples

### Materials & Methods

### Development of the Predictive Model:

The link between the response to the biotherapy and each variable is analysed through a logistic regression model on a set of data. The variable to explain is the good response after 6 months.

Firstly, a pre-selection of the variables to include in the multivariate model is carried out. To do so, the predictive capacity of each explanatory variable is analysed individually. The biomarkers are analysed in a quantitative and qualitative manner. A method for selecting variables is put in place to conserve uniquely the relevant variables that will next be introduced into a multivariate model. The biomarkers are preselected if they have:
In quantitative form, a p-value <0.25 or an AUC >0.60
In qualitative form, a p-value <0.05 and an AUC >0.65

The criteria chosen are voluntarily wide to include significant variables, but also variables presenting trends in the multivariate model.

The preselected biomarkers exhibit relevant trends to analyse. Multivariate models with the different possible combinations of these biomarkers are constructed, and the AUCs calculated. Models having an AUC >0.70 are considered as relevant and are conserved.

The models thereby constructed make it possible to weight the dosage results of each of the specific biomarkers to obtain a probability of response. The coefficients of each model make it possible to calculate from the dosage values of each patient an associated probability of response. The performance characteristics (AUC (area under curve), sensitivity and specificity, PPV (positive predictive value) and NPV (negative predictive value)) of each model are calculated to define its relevance.

An AUC >0.70 is considered as an acceptable discrimination, an AUC >0.80 demonstrates very good discrimination capacity. A level of probability threshold is fixed to calculate specificity and sensitivity. This optimal threshold is determined on the basis of the Youden index. At this threshold, patients may be classified as a function of the following table 1:

TABLE 1

| | Responder | Non-responder |
|---|---|---|
| Positive test (prob > threshold) | TP | FP |
| Negative test (prob < threshold) | FN | TN |

TP (true positives) represents the number of responder individuals with a positive test,
FP (false positives) represents the number of non-responder individuals with a positive test,
FN (false negatives) represents the number of responder individuals with a negative test,
TN (true negatives) represents the number of non-responder individuals with a negative test.
   The sensitivity, or the probability that the test is positive if the patient is a responder, is measured in sufferers only. It is given by:

$$\frac{T P}{T P + F N}$$

The specificity is measured in non-sufferers only. The specificity, or the probability of obtaining a negative test in non-responders is given by:

$$\frac{T N}{T N + F P}$$

The sensitivity of the test measures its capacity to provide a positive result when the patient is a responder. The specificity measures the capacity of the test to give a negative result when the patient is a non-responder.
The positive predictive value (PPV) is the probability that the patient is a responder when the test is positive.

$$P P V = \frac{T P}{T P + F P}$$

The negative predictive value (NPV) is the probability that the patient is a non-responder when the test is negative.

$$N P V = \frac{T N}{T N + F N}$$

### Results:

Out of a learning cohort (34 RA patients), the models constructed have the characteristics presented in table 1 below which shows the different combinations of 2 or 3 biomarkers with AUC >0.75. All the combinations including at least 2 of the 9 biomarkers among FetA, TBG, SAA, C4BP, S100A8A9, Lp(a), HAPT, C1q, CRP provide relevant results.

TABLE 2

| | | | | | | | | | | AUC |
|---|---|---|---|---|---|---|---|---|---|---|
| N | Feta | TBG | SAA | C4BP | S100A8A9 | Lp(a) | HAPT | C1q | CRP | Response |
| 3 | X | X | | | | X | | | | 0.93 |
| | X | | | X | | X | | | | 0.93 |
| | X | | X | | | X | | | | 0.93 |
| | X | X | | X | | | | | | 0.93 |
| | X | | | | | X | | X | | 0.97 |
| | | X | X | | | X | | | | 0.97 |
| | | X | | X | | X | | | | 0.96 |
| | | X | | | | X | | | X | 0.96 |
| | X | X | X | | | | | | | 0.96 |
| | X | | | | | X | X | | | 0.96 |
| | | | X | | | X | | X | | 0.96 |
| | X | | | | X | X | | | | 0.96 |
| | | X | | | | X | | X | | 0.95 |
| | X | | X | | | | | X | | 0.95 |
| | X | X | | | X | | | | | 0.95 |
| | | X | | | X | X | | | | 0.95 |
| | X | | | X | X | | | | | 0.95 |
| | X | | X | X | | | | | | 0.95 |
| | X | | | X | | | | X | | 0.95 |
| | X | | | | | X | | | X | 0.95 |
| | X | X | | | | | X | | | 0.95 |
| | | | | | | X | | X | X | 0.94 |
| | | X | X | | X | | | | | 0.94 |
| | X | | | X | | | | | X | 0.94 |
| | | X | X | | | | | | X | 0.94 |
| | X | X | | | | | | | X | 0.94 |
| | | | | X | | | | X | X | 0.94 |
| | | X | X | X | | | | | | 0.94 |
| | | X | X | | | | X | | | 0.94 |
| | X | | | X | | | X | | | 0.94 |
| | | X | X | | | | | X | | 0.94 |
| | X | X | | | | | | X | | 0.94 |
| | X | | | | | | | X | X | 0.93 |
| | | X | | | | X | X | | | 0.93 |
| | | X | | X | | | | | X | 0.93 |
| | X | | | | X | | | X | | 0.93 |
| | X | | X | | X | | | | | 0.93 |
| | | | X | | | | X | X | | 0.93 |
| | | | X | | | | | X | X | 0.93 |
| | | | | | | | X | X | X | 0.93 |
| | | | | | X | | | X | X | 0.91 |
| | | | X | X | | X | | | | 0.91 |
| | | X | | X | X | | | | | 0.90 |
| | | X | | X | | | | X | | 0.90 |
| | | | X | | | X | | | X | 0.90 |
| | X | | | | X | | | | X | 0.90 |
| | | | | | | X | X | X | | 0.90 |
| | | X | | | X | | X | | | 0.90 |
| | | | X | | X | | | X | | 0.39 |
| | X | | | | X | | X | | | 0.39 |
| | X | | | | | | X | | X | 0.39 |
| | | X | | | | | X | X | | 0.39 |
| | | | X | | X | X | | | | 0.39 |
| | | | | X | | X | | | X | 0.39 |
| | | | | X | X | X | | | | 0.39 |
| | | | | | | X | X | | X | 0.33 |
| | | | X | | | X | X | | | 0.33 |
| | | | X | X | | | | X | | 0.33 |
| | | | | X | | X | | X | | 0.33 |
| | | | X | X | | | | | X | 0.33 |
| | | | | | X | X | | | X | 0.33 |
| | | | X | X | X | | | | | 0.37 |
| | | | | X | X | | | X | | 0.37 |
| | | | X | X | | | X | | | 0.36 |
| | | | | X | | | X | X | | 0.36 |
| | | | | | X | X | X | | | 0.35 |
| | | | | X | | X | X | | | 0.35 |
| | | | | X | | | X | | X | 0.35 |
| | | | X | | X | | | | X | 0.35 |
| | | | | X | X | | | | X | 0.34 |
| | | | X | | X | | X | | | 0.34 |
| | | | X | | | | X | | X | 0.34 |
| | | | | | X | | X | X | | 0.34 |
| | | | | | X | | X | | X | 0.33 |
| | | | | X | X | | X | | | 0.30 |
| 2 | X | | | | | X | | | | 0.94 |
| | | X | X | | | | | | | 0.94 |
| | X | X | | | | | | | | 0.94 |
| | X | | | X | | | | | | 0.93 |
| | | X | | | | X | | | | 0.93 |
| | X | | X | | | | | | | 0.91 |
| | | X | | X | | | | | | 0.90 |
| | | X | | | | | | | X | 0.39 |
| | X | | | | X | | | | | 0.39 |
| | | X | | | X | | | | | 0.39 |
| | | | | | | | | X | X | 0.39 |
| | X | | | | | | X | | | 0.33 |
| | | | X | | | | | X | | 0.33 |
| | X | | | | | | | X | | 0.33 |
| | X | | | | | | | | X | 0.30 |
| | | | X | | | X | | | | 0.07 |
| | | X | | | | | X | | | 0.37 |
| | | | X | X | | | | | | 0.35 |
| | | | | | | X | | | X | 0.35 |
| | | | | | X | X | | | | 0.05 |
| | | | | X | | | | | X | 0.04 |
| | | | X | | X | | | | | 0.33 |
| | | | X | | | | | | X | 0.33 |
| | | | | | | X | X | | | 0.33 |
| | | | | X | | X | | | | 0.03 |
| | | | X | | | | X | | | 0.02 |
| | | | | | X | | | X | | 0.01 |
| | | | | | | | X | | X | 0.01 |
| | | | | X | | | | X | | 0.30 |
| | | | | | X | | | | X | 0.30 |
| | | X | | | | | | X | | 0.30 |
| | | | | X | X | | | | | 0.79 |
| | | | | | | X | | X | | 0.79 |
| | | | | X | | | X | | | 0.70 |
| | | | | | | | X | X | | 0.77 |
| | | | | | X | | X | | | 0.75 |

Taking for example the model with 3 variables Fet-A, TBG and Lp(a) the characteristics obtained are presented in table 3 below.

TABLE 3
Model with 3 variables: Fet-A, TBG and Lp(a)

| | Response |
|---|---|
| AUC | 0.98 |
| Sensitivity | 1 |
| Specificity | 0.96 |
| PPV | 0.91 |
| NPV | 1 |

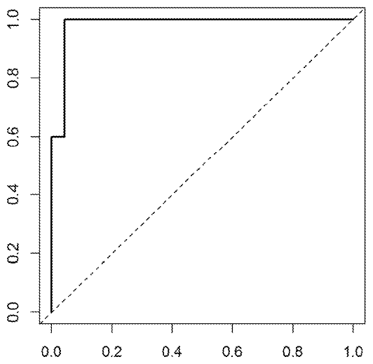

The corresponding ROC curve is represented in appended FIG. 1.

### REFERENCES

Amos, R. S, Constable, T. J, Crockson, R. A, Crockson, A. P., and McConkey, B. (1977). Rheumatoid arthritis: relation of serum C-reactive protein and erythrocyte sedimentation rates to radiographic changes. Br. Med. J. 1, 195-197.
Andersen, C. B. F., Stødkilde, K., Sæderup, K. L, Kuhlee, A., Raunser, S., Graversen, J. H., and Moestrup, S. K (2017). Haptoglobin. Antioxid. Redox Signal. 26, 814-831.
Baillet, A. (2010). [S100A8, S100A9 and S100A12 proteins in rheumatoid arthritis]. Rev. Med. Interne 31, 458-461.
Baillet, A., Trocmé, C., Berthier, S., Arlotto, M., Grange, L., Chenau, J., Quétant, S., Sève, M., Berger, F., Juvin, R., et al. (2010). Synovial fluid proteomic fingerprint: S100A8, S100A9 and S100A12 proteins discriminate rheumatoid arthritis from other inflammatory joint diseases. Rheumatol. Oxf. Engl. 49, 671-682.
Blaschke, S., Rinke, K., Maring, M., Flad, T., Patschan, S., Jahn, O., Mueller, C. A, Mueller, G. A, and Dihazi, H. (2015). Hatoglobin-α1, -α2, vitamin D-binding protein and apolipoprotein C-III as predictors of etanercept drug response in rheumatoid arthritis. Arthritis Res. Ther. 17.
Blom, A. M, Nandakumar, K. S., and Holmdahl, R. (2009). C4b-binding protein (C4BP) inhibits development of experimental arthritis in mice. Ann. Rheum. Dis. 68, 136-142.
Choi, I. Y, Gerlag, D. M, Herenius, M. J, Thurlings, R. M, Wijbrandts, C. A, Foell, D., Vogl, T., Roth, J., Tak, P. P., and Holzinger, D. (2015a). MRP8/14 serum levels as a strong predictor of response to biological treatments in patients with rheumatoid arthritis. Ann. Rheum. Dis. 74, 499-505.
Choi, I. Y, Gerlag, D. M, Herenius, M. J, Thurlings, R. M, Wijbrandts, C. A, Foell, D., Vogl, T., Roth, J., Tak, P. P., and Holzinger, D. (2015b). MRP8/14 serum levels as a strong predictor of response to biological treatments in patients with rheumatoid arthritis. Ann. Rheum. Dis. 74, 499-505.
Dabrowska, A. M, Tarach, J. S, Wojtysiak-Duma, B., and Duma, D. (2015). Fetuin-A (AHSG) and its usefulness in clinical practice. Review of the literature. Biomed. Pap. Med. Fac. Univ. Palacky Olomouc Czechoslov. 159, 352-359.
Drynda, S., Ringel, B., Kekow, M., Kühne, C., Drynda, A., Glocker, M. O, Thiesen, H.-J., and Kekow, J. (2004). Proteome analysis reveals disease-associated marker proteins to differentiate RA patients from other inflammatory joint diseases with the potential to monitor anti-TNFalpha therapy. Pathol. Res. Pract. 200, 165-171.
Gabay, C., McInnes, I. B, Kavanaugh, A., Tuckwell, K., Klearman, M., Pulley, J., and Sattar, N. (2016). Comparison of lipid and lipid-associated cardiovascular risk marker changes after treatment with tocilizumab or adalimumab in patients with rheumatoid arthritis. Ann. Rheum. Dis. 75, 1806-1812.
Harman, H., Tekeo\check{g}lu, İ, Gürol, G., Sa\check{g}, M. S, Karakeçe, E., Ç\dot{l}ftç\dot{l}, İ. H, Kamanli, A., and Nas, K. (2017). Comparison of fetuin-A and transforming growth factor beta 1 levels in patients with spondyloarthropathies and rheumatoid arthritis. Int. J. Rheum. Dis. 20, 2020-2027.
Isaacs, J. D, Cohen, S. B, Emery, P., Tak, P. P, Wang, J., Lei, G., Williams, S., Lal, P., and Read, S. J (2013). Effect of baseline rheumatoid factor and anticitrullinated peptide antibody serotype on rituximab clinical response: a meta-analysis. Ann. Rheum. Dis. 72, 329-336.
Nair, S. C, Welsing, P. MJ., Choi, I. YK., Roth, J., Holzinger, D., Bijlsma, J. WJ., van Laar, J. M, Gerlag, D. M, Lafeber, F. P. J. G., and Tak, P. P (2016). A Personalized Approach to Biological Therapy Using Prediction of Clinical Response Based on MRP8/14 Serum Complex Levels in Rheumatoid Arthritis Patients. PloS One 11.
Orsó, E., and Schmitz, G. (2017). Lipoprotein(a) and its role in inflammation, atherosclerosis and malignancies. Clin. Res. Cardiol. Suppl. 12, 31-37.
Papichev, E. V, Zavodovsky, B. V, Polyakova, Y. V, Seewordova, L. E., and Akhverdyan, Y. R (2018). [Novel hepatokine in rheumatoid arthritis laboratory diagnostics.]. Klin. Lab. Diagn. 63, 756-760.
Patro, P. S, Singh, A., Misra, R., and Aggarwal, A. (2016). Myeloid-related Protein 8/14 Levels in Rheumatoid Arthritis: Marker of Disease Activity and Response to Methotrexate. J. Rheumatol. 43, 731-737.
Persellin, R. H., and Cheng, C. T (1981). Serum C1q concentrations in rheumatic disorders. Early normalization during treatment of immunologically-mediated vasculitis. Am. J. Clin. Pathol. 76, 462-466.
Raterman, H. G, Vosslamber, S., de Ridder, S., Nurmohamed, M. T, Lems, W. F, Boers, M., van de Wel, M., Dijkmans, B. AC., Verweij, C. L., and Voskuyl, A. E (2012). The interferon type I signature towards prediction of non-response to rituximab in rheumatoid arthritis patients. Arthritis Res. Ther. 14, R95.
Raterman, H. G, Levels, H., Voskuyl, A. E, Lems, W. F, Dijkmans, B. A., and Nurmohamed, M. T (2013). HDL protein composition alters from proatherogenic into less atherogenic and proinflammatory in rheumatoid arthritis patients responding to rituximab. Ann. Rheum. Dis. 72, 560-565.
Refetoff, S., Murata, Y., Mori, Y., Janssen, O. E., Takeda, K., and Hayashi, Y. (1996). Thyroxine-binding globulin: organization of the gene and variants. Horm. Res. 45, 128-138.
Ricklin, D., and Lambris, J. D (2013). Complement in immune and inflammatory disorders: therapeutic interventions. J. Immunol. Baltim. Md. 1950 190, 3839-3847.
Saroha, A., Biswas, S., Chatterjee, B. P., and Das, H. R (2011). Altered glycosylation and expression of plasma alpha-1-acid glycoprotein and haptoglobin in rheumatoid arthritis. J. Chromatogr. B Analyt. Technol. Biomed. Life. Sci. 879, 1839-1843.
Sellam, J., Rivière, E., Courties, A., Rouzaire, P.-O., Tolusso, B., Vital, E. M, Emery, P., Ferraccioli, G., Soubrier, M., Ly, B., et al. (2016). Serum IL-33, a new marker predicting response to rituximab in rheumatoid arthritis. Arthritis Res. Ther. 18, 294.
Seriolo, B., Paolino, S., SuIli, A., Fasciolo, D., and Cutolo, M. (2006). Effects of anti-TNF-alpha treatment on lipid profile in patients with active rheumatoid arthritis. Ann. N. Y. Acad. Sci. 1069, 414-419.
Swaak, A. J, Van Rooyen, A., Planten, O., Han, H., Hattink, O., and Hack, E. (1987). An analysis of the levels of complement components in the synovial fluid in rheumatic diseases. Clin. Rheumatol. 6, 350-357.
Takeuchi, T., Nakanishi, T., Tabushi, Y., Hata, A., Shoda, T., Kotani, T., Shimizu, A., Takubo, T., Makino, S., and Hanafusa, T. (2007). Serum protein profile of rheumatoid arthritis treated with anti-TNF therapy (infliximab). J. Chromatogr. B Analyt. Technol. Biomed. Life. Sci. 855, 66-70.
Tan, W., Wang, F., Guo, D., Ke, Y., Shen, Y., Lv, C., and Zhang, M. (2016). High serum level of haptoglobin is associated with the response of 12 weeks methotrexate therapy in recent-onset rheumatoid arthritis patients. Int. J. Rheum. Dis. 19, 482-489.
Tony, H.-P., Roll, P., Mei, H. E, Blümner, E., Straka, A., Gnuegge, L., Dörner, T., and FIRST/ReFIRST study teams (2015). Combination of B cell biomarkers as independent predictors of response in patients with rheumatoid arthritis treated with rituximab. Clin. Exp. Rheumatol. 33, 887-894.
Trouw, L. A, Pickering, M. C., and Blom, A. M (2017). The complement system as a potential therapeutic target in rheumatic disease. Nat. Rev. Rheumatol. 13, 538-547.
Wells, G., Becker, J.-C., Teng, J., Dougados, M., Schiff, M., Smolen, J., Aletaha, D., and van Riel, P. L. C. M. (2009). Validation of the 28-joint Disease Activity Score (DAS28) and European League Against Rheumatism response criteria based on C-reactive protein against disease progression in patients with rheumatoid arthritis, and comparison with the DAS28 based on erythrocyte sedimentation rate. Ann. Rheum. Dis. 68, 954-960.
Wouters, D., Voskuyl, A. E, Molenaar, E. TH., Dijkmans, B. A. C., and Hack, C. E (2006). Evaluation of classical complement pathway activation in rheumatoid arthritis: measurement of C1q-C4 complexes as novel activation products. Arthritis Rheum. 54, 1143-1150.

1.-15. (canceled)

16. Method for estimating the effectiveness of treatment with an anti-CD20 agent in a patient with rheumatoid arthritis and who has had an inadequate response to at least one prior biotherapy treatment, the method comprising:
a) an in vitro measurement of an expression level of at least two biomarkers chosen from a group of biomarkers consisting of Fetuin-A (FetA), Thyroxine Binding Globulin (TBG), Serum Amyloid A Protein (SAA), C4b Binding Protein (C4BP), Protein complex S100A8/A9 (S100A8A9), Lipoprotein(a) (Lp(a)), Haptoglobin (HAPT), Complement protein C1q (C1q), and C-reactive protein (CRP), in a biological sample from the patient; and
b) an estimation of an effectiveness of treatment with the anti-CD20 agent in the patient as a function of each expression level measured for the biomarkers chosen from the group of biomarkers.

17. Method for estimating the effectiveness of treatment with an anti-CD20 agent in a patient with rheumatoid arthritis and who has had an inadequate response to at least one prior biotherapy treatment, the method comprising:
a) an in vitro measurement of an expression level of at least two biomarkers chosen from a group of biomarkers consisting of Fetuin-A (FetA), Thyroxine Binding Globulin (TBG), Serum Amyloid A Protein (SAA), C4b Binding Protein (C4BP), Protein complex S100A8/A9 (S100A8A9), Lipoprotein(a) (Lp(a)), Haptoglobin (HAPT), Complement protein C1q (C1q), and C-reactive protein (CRP), in a biological sample from the patient;
b1) a comparison of the expression level measured at step a) compared to that measured in a plurality of samples of patients with rheumatoid arthritis and having received a treatment with the anti-CD20 agent for which the effectiveness of treatment is known; the comparison being carried out by means of a statistical learning model using as input data the expression levels of at least two of the biomarkers measured at step a), b2) an estimation of an effectiveness of treatment with the anti-CD20 agent in the patient as a function of the results determined by the model defined at step b1).

18. Method for estimating the effectiveness of treatment with an anti-CD20 agent according to claim 16, wherein the expression levels of each biomarker measured at step a) are used to obtain a score linked to the estimation of the effectiveness of treatment in the patient, the score being compared with at least one predetermined threshold so as to classify the prognosis among a plurality of classes.

19. Method for estimating the effectiveness of treatment with an anti-CD20 agent according to claim 18, wherein the plurality of classes comprises at least two classes of which a class of non-response to the treatment with the anti-CD20 agent.

20. Method for estimating the effectiveness of treatment with an anti-CD20 agent according to claim 18, wherein the estimation of the effectiveness of treatment in the patient comprises the comparison of the score with a predetermined threshold below which poor effectiveness is predicted and above which good effectiveness is predicted.

21. Method for estimating the effectiveness of treatment with an anti-CD20 agent according to claim 17, wherein the learning model is based on a prior analysis of a cohort comprising patients treated with the anti-CD20 agent presenting good responses to the treatment and patients treated with the anti-CD20 agent presenting poor responses to the treatment.

22. Method for estimating the effectiveness of treatment with an anti-CD20 agent according to claim 21, wherein the prior analysis comprises an application of a method for learning and selecting variables.

23. Method for estimating the effectiveness of treatment with an anti-CD20 agent according to claim 22, wherein the method for learning and selecting variables is a logistic regression.

24. Method for estimating the effectiveness of treatment with an anti-CD20 agent according to claim 21, wherein the expression levels are weighted as a function of the prior analysis of the cohort to derive a score.

25. Method for estimating the effectiveness of treatment with an anti-CD20 agent according to claim 22, wherein the method for learning comprises a decision tree wherein each node corresponds to a comparison of the expression level measured at step a) with a reference value.

26. Method for estimating the effectiveness of treatment with an anti-CD20 agent according to claim 16, wherein step b) relates to the estimation of the effectiveness of treatment with the agent Rituximab.

27. Method for estimating the effectiveness of treatment with an anti-CD20 agent according to claim 16, wherein the biological sample in which the in vitro measurement of the expression level of at least two biomarkers chosen from the group that is carried out at step a) is from a patient who has had an inadequate response to at least one prior treatment chosen from among Etanercept, Adalimumab, Infliximab, Tocilizumab, Abatacept, Certolizumab Pegol, Golimumab, Sarilumab, and Anakinra.

28. Method for estimating the effectiveness of treatment with an anti-CD20 agent according to claim 16, wherein the biological sample is constituted of a sample of biological fluid.

29. Method for estimating the effectiveness of treatment with an anti-CD20 agent according to claim 16, characterised in that the biomarker(s) of which the expression level is measured at step a) is a/are protein biomarker(s).

30. System for estimating the effectiveness of treatment with an anti-CD20 agent in a patient with rheumatoid arthritis and who has had an inadequate response to at least one prior biotherapy treatment, the system comprising:
means for measuring or receiving measurement data of an expression level of at least two biomarkers chosen from a group of biomarkers consisting of: Fetuin-A (FetA), Thyroxine Binding Globulin (TBG), Serum Amyloid A protein (SAA), C4b Binding Protein (C4BP), Protein complex S100A8/A9 (S100A8A9), Lipoprotein(a) (Lp(a)), Haptoglobin (HAPT), Complement protein C1q (C1q), and C-reactive protein (CRP) in a biological sample from the patient; and
means for processing measurement data configured to estimate the effectiveness of treatment by the anti-CD20 agent in the patient as a function of each expression level measured for the biomarkers chosen from the group of biomarkers.

31. Method for estimating the effectiveness of treatment with an anti-CD20 agent according to claim 16, wherein the biological sample is serum.
